(19) United States
(12) Patent Application Publication
(10) Pub. No.: US 2019/0150770 A1
(43) Pub. Date: May 23, 2019

(54) DIAGNOSTIC SYSTEM

(71) Applicant: Standard Instruments GmbH, Karlsruhe (DE)

(72) Inventor: Andreas Schoenfeld, Karlsruhe (DE)

(73) Assignee: Standard Instruments GmbH, Karlsruhe (DE)

(21) Appl. No.: 16/320,044

(22) PCT Filed: Jul. 25, 2017
(86) PCT No.: PCT/EP2017/068794

§ 371 Date: Jan. 23, 2019

(30) Foreign Application Priority Data
Jul. 25, 2016 (DE) 10 2016 113 657.3

(51) Int. Cl. A61B 5/042; A61B 5/03; A61B 5/053; A61B 5/0488; A61B 5/22

(52) U.S. Cl. CPC A61B 5/0421; A61B 5/037; A61B 5/04325; A61B 5/04884; A61B 5/222; A61B 5/0538

(57) ABSTRACT

The invention relates to a diagnostic system for the simultaneous long-term registration of heart rate variability as well as the pressure value and/or the impedance and, if so desired, further measured values of a patient with a measuring probe system suitable for deriving data for electrocardiogram generation, a catheter for insertion into the esophagus of the patient, the catheter being provided with several measuring probes each for pressure determination and/or for impedance measurement, and, if required, further measuring probes for determining further measured values, a recording apparatus having a voltage source and a plurality of channels for the registration and recording of the measurement data originating from the measuring probes, at least one memory for time-dependent storage of the measurement data and means for data transmission from the measuring probes to the recording apparatus for use in the diagnosis of motility disorders influenced by the autonomic nervous system and the use of this system in the differential diagnosis of motility disorders.

The invention relates to a diagnostic system, by means of which during a long-term registration process measured values from a patient's esophagus as well as their heart rate variability are determined with a view to obtaining information about the condition of the patient's autonomic nervous system.

Inexplicable complaints in the upper abdomen can have cardiological, orthopedic or gastroenterological causes. In the case of gastroenterological causes, the focus of current diagnostic efforts is on clarifying motility disorders. Such motility disorders can generally be attributed to pressure waves arising in a non-coordinated fashion. In addition, functional disorders of the sphincters are causally involved as a rule. For example, more often than not an incompetent lower esophageal sphincter leads to complaints known as refluxes (heartburn in colloquial speech).

The investigation of motility disorders is currently carried out using methods such as manometry, impedance measurement in the esophagus for the representation of an orderly transport and pH-metry for the representation of acidic refluxate. This also allows collection of symptom-related evidence of the involvement of motility disorders in the complaints.

If a participation of the motility disorders is now diagnosed as the reason for the complaints, the question arises which therapy is the best. This is exactly where the invention comes in and provides the attending physician with another option for differential diagnosis.

Aside from organic damage, misplacement and lesions, the involvement of the autonomic nervous system plays a not to be underestimated role in the system's functioning, since it modulates the functions of the individual muscle groups by influencing the otherwise independent nervous system of the digestive tract, the enteric nervous system, via both sympathicus and parasympathicus. In order to enable further research into the causes of the malfunctions, it is essential to determine/record the activities of the autonomic nervous system.

The activity of the autonomic nervous system cannot be readily acquired/measured directly, but there are some possibilities for non-invasive acquisition: Conductivity measurements of the skin, blood pressure measurements, pupillary light reflex, respiratory rate measurements or—particularly suitable—heart rate variability. In addition to the activity-related heart rate, there is a further influence on the heart rate through psychological stress, which can be represented by recording the heart rate variability.

If, in addition to the classical diagnostic methods, the simultaneous recording of heart rate variability is used to assess the causes of symptoms, this may, under certain circumstances, decisively change the decision which form of therapy should be adopted.

Heart rate variability is preferably measured by recording the change in the time occurring between two heartbeats, usually represented by the so-called R-wave, from the heartbeats derived by an ECG and presenting it in a suitable form with the other data that have been determined. The representation can be displayed in the form of a simple derivation of the temporal progress or as a frequency spectrum.

In the presentation of heart rate variability, it is also expedient to take the correlation with respiration into account, as this correlates with heart rate variability especially in a relaxed state. Breathing data can be extracted from pressure data measured in the area of the stomach. Breathing causes the basic pressure in the stomach to change from rising when inhaling to falling when exhaling. If the correlation with respiration cannot be verified, this is an indication of an increased stress burden acting on the patient.

The finding that symptoms are associated with classic motility disorders, possibly manifested by refluxes, and at the same time high stress levels, supports the consideration that surgical intervention in the sphincter area may not lead to the desired success and that other methods may possibly have to be given preference.

State of the art measuring probes are known for the acquisition of motility data in the esophagus. Measuring catheters for the determination of impedance values, pH values and pressure values and the combination of such a measuring catheter with ECG data simultaneously derived from the patient are known for example from U.S. Pat. No. 4,503,859 A and DE 38 36 349 A1 publications. However, there is no indication in these publications that say that the activity of the autonomic nervous system can be responsible for the cause of a motility disorder and that the activity of the autonomic nervous system can be derived from an ECG.

It is thus the objective of the present invention to provide a diagnostic apparatus that is suitably applied to clarify non-specific pain in the upper abdomen and chest area and examine an underlying motility disorder of the esophagus with a view to finding out whether this can be associated with the state of the autonomic nervous system.

This objective is achieved with a diagnostic system for the simultaneous long-term registration of heart rate variability, pressure and impedance data from the esophagus of a patient, said system comprising
a measuring probe system suitable for establishing the electrocardiogram,
a catheter for insertion into the patient's esophagus, the catheter being provided with several measuring probes each for measuring pressure and impedance,
a recording device with a voltage source and a plurality of channels for registration and recording of the measurement data collected by means of the measuring probes, and at least one memory system for time-dependent storage of the measurement data, and
means for data transmission from the measuring probes to the recording device,
wherein the diagnostic system being intended for use in the differential diagnosis of motility disorders influenced by the autonomic nervous system.

As a rule, the diagnostic system has several measuring probes each for manometry and impedance measurement. It can have additional measuring probes, for example for determining the pH value and other data.

The diagnostic system proposed by the invention is designed to simultaneously register and record an electrocardiogram as well as data relevant for the diagnosis of a motility disorder. This enables two examinations to be carried out in parallel which are conducive to diagnosing on the basis of heart rate variability a possible influence of the autonomic nervous system on motility disorders.

As far as the electrocardiogram is concerned, reference can be made to the conventional technique to be adopted for recording long-term ECGs. In particular, the signals are derived via chest leads, as this restricts the patient's freedom of movement only slightly. However, signal derivation may also take place at the extremities or directly in the esophagus. It may be bipolar or unipolar.

The catheter, especially the nasal catheter, is provided with several measuring probes to determine the impedance. On the basis of recording changes in electrical conductivity, impedance measurement makes it possible, in particular, to detect movements of masses in the esophagus that cannot be detected via the pH electrodes. This applies in particular to the penetration of acid-poor stomach contents into the esophagus—in this case the typical symptoms of sour eructation and heartburn are absent. At the same time, however, anomalies in drinking and swallowing, the peristalsis, can be diagnosed and recorded.

For example, this is also important when patients are treated with acid blockers or proton pump inhibitors, said treatment significantly reducing the acidity of the reflux. Nevertheless, these patients, who may otherwise be free of complaints, still suffer from reflux rather frequently, which can be easily detected in this manner.

As a rule, ring electrodes placed around the catheter are employed for impedance measurement. Ring electrodes of this kind are known per se.

Additionally, the inventive diagnostic system also features several pressure sensors. With the help of the so-called esophageal manometry, it is possible to diagnose coordination disorders and measure the pressure conditions prevailing in the esophageal muscles, particularly in the case of difficulty swallowing or painful passage of food into the stomach. In particular, malfunctions of the sphincter muscles can also be diagnosed.

For the collection of data from the patient's esophagus a catheter is used, preferably a nasal catheter, which usually causes the patient little discomfort. Such a nasal catheter can be of a very small diameter, for example 1.5 mm, so that it practically does not interfere with the intake of food and fluid.

The nasal catheter is preferably equipped with at least one, usually two pH-sensitive electrodes, one of which is located approximately 5 cm away from the distal end of the catheter. The distal end of the catheter is the end pointing into the interior of the body, as opposed to the proximal end which points to the recording device and serves to control and guide the connection between the probe and the recording device, unless data transmission is accomplished via telemetry.

In practice, the catheter is arranged such that a pH measuring point is located about 5 cm above the cardia. The position on the catheter can be arbitrary. Actually existing catheters are often provided with a reference electrode located at the distal end, in which case the measuring point lies above it.

A second pH-sensitive probe may be located at the distal end of the catheter, which causes it to regularly come into contact with the gastric acid itself and take a comparative value measurement there. As a rule, however, this second pH probe will be arranged proximal to the first probe and take a reading in the middle or upper part of the esophagus. More than two pH-sensitive probes may be provided to determine the path of the reflux bolus in the esophagus.

The pH-sensitive probes are customary glass, ISFET or antimony electrodes as they have been in use for pH registration in the body for a long time.

With regard to pH measurements and in the event pH probes are provided, the recording device is designed so that it can be calibrated, that is, adapted both to the type of probe and the age condition of the probe. It is known that the measurement characteristics of pH probes change with age and frequency of use and therefore probe calibration is necessary.

It is also useful and expedient if the calibration and/or measurement temperature can be set on the recording device.

The pH electrodes are reliably suitable for detecting the penetration or ingress of acidic stomach contents into the esophagus. If several probes are arranged along the length of the catheter, the rise of gastric acid can also be registered over the length of the esophagus, which is of significance especially in the case of patients who are lying down.

Additionally, the inventive system can also be provided with one or more EMG probes, by means of which intra-esophageal muscle actions can be recorded independently of the pressure generated.

Moreover, the catheter may also be provided with a probe for the detection of enzymes, such as those secreted in the small intestine or duodenum. This “small intestinal juice” also frequently exits from the small intestine and enters the stomach from where it may enter the esophagus together with the reflux mass. Since this juice contains highly effective digestive enzymes, they may further damage the already severely damaged mucous membrane of the esophagus.

Alternatively or cumulatively to the ECG probes, the system according to the invention can additionally be provided with probes for EEG measurements, especially those for the derivation from the cortex.

The recording device itself is provided with a customary voltage source, for example a battery or rechargeable battery, and the necessary channels required for the registration and recording of the measured data. Furthermore, in addition to at least one mass storage device, preferably a memory card, an additional memory unit for programming the device functions is provided for the time-dependent storage of the measured values.

In addition, the system is equipped with the necessary connections/leads between the recording device and the measurement probes for power supply and data transmission purposes. It is expressly pointed out that data can also be transmitted wirelessly (telemetrically), for example via RFID systems. Therefore, means for data transmission between measuring probes and recording device are all connections that are suitable for signal transmission, be they via lines or wireless.

For registration of the measured data, conventional recording devices can basically be used, as they are often described in relevant literature. Aside from permanent storage capability for the measuring program such a recording device should, however, in particular be provided with separate memory cards for recording the ECG on the one hand and the measurement data obtained from the esophagus on the other. After completion of the examination, these memory cards can be read out and evaluated via a conventional PC and subsequently reconfigured for the next patient.

Moreover, the recording device should be programmable in terms of duration and/or with respect to the intervals of the measurements. This applies in particular to the time periods over which the heart function is recorded and causes considerably more data to be produced than with the punctual measured values which are recorded from time to time from the esophagus. As regards the pH data collected from the esophagus, it is usually sufficient to take a measurement every 4 to 20 seconds, impedance measurements are usually recorded at a rate of 50 measurements per second.

Preferably, the recording device can also be adjusted to the respective type of measuring probe, i.e. applicable characteristics of customary measuring probes are stored in the recording device and can be called up at any time for a quick connection.

The system proposed by the invention allows parallel diagnostics of heart rate variability and thus of the activity of the autonomic nervous system, as well as comprehensive diagnostics of the processes that take place in the esophagus of a patient over a longer period of time, for example 24 hours, but also for a longer time. The arrangement of several measuring probes in the esophageal catheter not only enables the masses ingressing into the esophagus to be detected, but also allows the direction of movement and, if so desired, the behavior of the esophageal muscles to be determined. In particular, it is possible to measure the direction of transport and the height up to which the reflux rises. The reflux can be determined independently of its acid content.

The invention furthermore relates to the use of the above described diagnostic system for the simultaneous long-term registration of heart rate variability as well as pressure and/or impedance data collected from a patient's esophagus for the diagnosis of motility disorders influenced by the autonomic nervous system. In particular, this enables a differential diagnosis of motility disorders to be performed that are caused by the autonomic nervous system as well as motility disorders attributable to organic factors, such as those having gastroenterological causes.

1. Diagnostic system in which, in addition to pressure and/or impedance data collected from the esophagus of a patient, the patient's heart rate variability is recorded over a long-term registration period, said system comprising
a measuring probe system suitable for recording the electrocardiogram,
a catheter for insertion into the patient's esophagus, the catheter being provided with several measuring probes each for measuring pressure and/or impedance,
a recording device with a voltage source and a plurality of channels for registration and recording of the measurement data collected by means of the measuring probes, and at least one memory system for time-dependent storage of the measurement data, and
means for data transmission from the measuring probes to the recording device
for use in the differential diagnosis of motility disorders.

2. Diagnostic system according to claim 1, characterized by a system of electrodes for ECG recording via chest lead derivation.

3. Diagnostic system according to claim 1, characterized in that the catheter is a nasal catheter.

4. Diagnostic system according to claim 1, characterized by at least one pH-sensitive electrode.

5. Diagnostic system according to claim 4 characterized by a glass, ISFET or an antimony electrode.

6. Diagnostic system according to claim 4, characterized in that one of the pH-sensitive electrodes is located about 5 cm away from the distal end of the catheter.

7. Diagnostic system according to claim 4, characterized in that the recording device can be calibrated with respect to the pH measurements.

8. Diagnostic system according to claim 4, characterized in that calibration and/or measuring temperature are adjustable on the recording device.

9. Diagnostic system according to claim 1, characterized in that the measuring probes for impedance measurement are ring electrodes.

10. Diagnostic system according to claim 1, characterized in that the recording device is provided with separate memory cards for recording the electrocardiogram and collection of the measurement data from the esophagus.

11. Diagnostic system according to claim 1, characterized in that said system is programmable with respect to the duration and/or the time sequence of the measurement data.

12. Diagnostic system according to claim 1, characterized in that the recording device is adjustable to the respective type of measuring probe.

13. Diagnostic system according to claim 11, characterized in that said system is preprogrammed for customary measuring probe systems.

14. Diagnostic system according to claim 1, characterized in that wireless data transmission takes place between measuring probes and the recording apparatus.

15. Use of the diagnostic system according to claim 1 for the simultaneous long-term registration of heart rate variability as well as pressure and/or impedance data collected from a patient's esophagus for the diagnosis of motility disorders influenced by the autonomic nervous system.

16. Use according to claim 15 in differential diagnostics for distinguishing motility disorders caused by the autonomic nervous system from motility disorders that are attributable to organic causes.
